(19) United States
(12) Patent Application Publication
(10) Pub. No.: US 2020/0222459 A1
(43) Pub. Date: Jul. 16, 2020

(54) System, Method and Compositions for Treating Age-Related Illnesses and Conditions

(71) Applicant: Melvin Barnes, Henderson, NV (US)

(72) Inventor: Melvin Barnes, Henderson, NV (US)

(21) Appl. No.: 16/742,675

(22) Filed: Jan. 14, 2020

Related U.S. Application Data
(60) Provisional application No. 62/793,298, filed on Jan. 16, 2019.

(51) Int. Cl. A61K 35/16; A61K 38/38; A61K 38/18

(52) U.S. Cl. CPC A61K 35/16; A61K 38/1841; A61K 38/38

(57) ABSTRACT

A system, method and compositions for treating age-related illnesses and conditions is provided. In one embodiment a kit comprises two or more containers, wherein each container comprises a young donor plasma product and indicia comprising information related to the age of donors of the young donor plasma product and wherein the young donor plasma product is sourced from a plurality of donors. The plurality of donors are within an age range at the time of donation all male. The age range includes an upper limit of 25 years of age or less and the lower limit of 16 years of age or more. The young donor plasma product has been processed to remove fractions having an average molecular weight below a predetermined threshold.

### CROSS REFERENCE TO RELATED APPLICATIONS

This application claims priority to U.S. Provisional Patent Appl. No. 62/793,298 filed Jan. 16, 2019, which is hereby incorporated herein by reference in its entirety for all purposes.

### BACKGROUND OF THE INVENTION

As the average lifespan of humans has lengthened, more and more people suffer from age-related conditions and illness. Aging is a primary or significant risk factor in numerous illnesses including but not limited to dementia (including Alzheimer's disease), atherosclerosis, cardiovascular disease, cancer, arthritis, hypertension, orthostatic hypotension, frailty syndrome, urinary tract infection, hip fracture, bacteremia, urinary incontinence, osteoporosis, influenza, heart rhythm problems, hypothyroidism, Parkinson's disease, eye disease (e.g., glaucoma, macular degeneration, cataracts), enlarged prostate, asthma, insomnia, fatigue, type 2 diabetes, etc.

Physiological changes beyond diagnosed disease also occur with aging. Typically, in humans cardiac output decreases, blood pressure increases, and arteriosclerosis develops. The lungs have impaired gas exchange, a decrease in vital capacity and slower expiratory flow rates, and creatinine clearance decreases. In addition, progressive elevation of blood glucose occurs with age as does the likelihood of osteoporosis. The epidermis of the skin atrophies with age and (due to changes in collagen and elastin) the skin loses its tone and elasticity. Lean body mass declines with age due primarily due to loss and atrophy of muscle cells. Degenerative changes occur in many joints and this, combined with the loss of muscle mass, inhibits elderly person's movement, which can result in further physiological and health declines. (Boss, Gerrry R.; Seegmiller J. Edwin, Age-Related Physiological Changes and Their Clinical Significance, West J. Med.; 135(6) 434-440 (1981 December)). In other words, although not always diagnosed clinically, the aged often suffer from numerous age-related conditions (or symptoms) that are typically thought of as normal for someone growing old such as low energy, muscle weakness, graying hair, baldness, diminished hearing, diminished eyesight, muscle pains, joint pain, shortness of breath, insomnia, incontinence, etc.

Aging, whether considered the natural progression of an organism through life or a disease, is one hundred percent fatal. The fatality is either a direct result of aging or an indirect result due to the illnesses and/or conditions largely dependent on aging.

The rate of aging in humans and other organisms was once thought to be constant and immutable. However, that may not be the case as numerous animal studies have demonstrated that it is possible to increase an animal's lifespan. Most recently, human studies have indicated the same.

Successfully slowing or reversing the physiological effects of aging will confer a broad spectrum of benefits on the patient—especially in comparison to treating a single age-related condition/symptom or illness. Consequently, there is a need to slow aging and/or to reverse the effects of aging of the human physiology. There also is a need to increase the health span of humans to more closely match the human lifespan as it increases to thereby reduce the likelihood of a person from suffering from age-related conditions and/or illnesses (even if such conditions or illnesses are not themselves fatal). Some embodiments of the present invention provide means for satisfying one or more of these needs and/or providing other solutions.

### BRIEF DESCRIPTION OF THE DRAWINGS

The invention is further described in the detailed description that follows, by reference to the noted drawings by way of non-limiting illustrative embodiments of the invention, in which like reference numerals represent similar parts throughout the drawings. As should be understood, however, the invention is not limited to the precise arrangements and instrumentalities shown.

FIG. 1 is an illustration of kit according to an example embodiment of the present invention; and

FIG. 2 is an illustration of method of an example embodiment of the present invention.

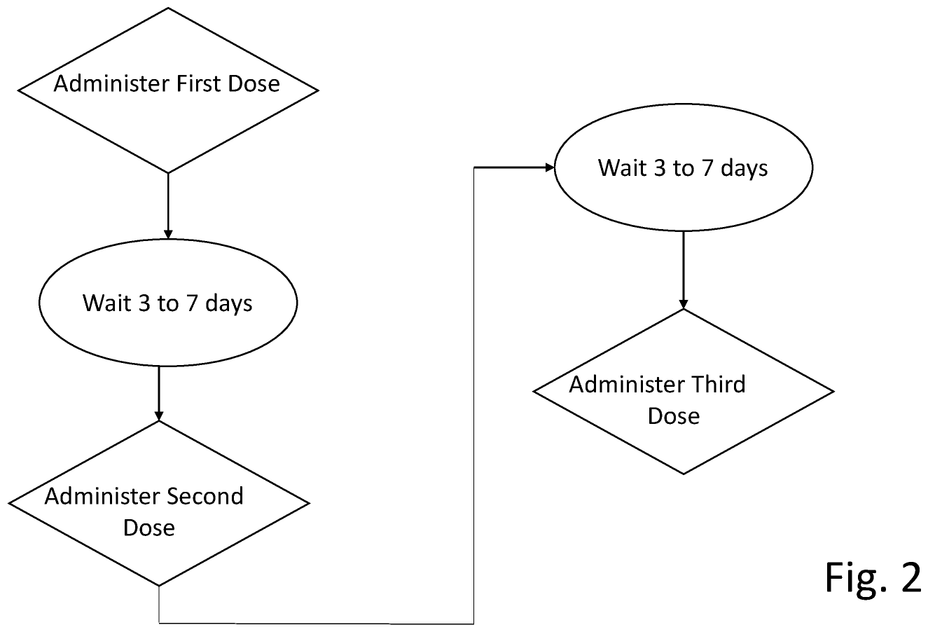

### DETAILED DESCRIPTION OF ILLUSTRATIVE EMBODIMENTS

In the following description, for purposes of explanation and not limitation, specific details are set forth. It should be understood that this invention is not limited to a particular method or composition described, as such may, of course, vary and is provided in order to provide a thorough understanding of the present invention. It should also be understood that the terminology used herein is for the purpose of describing particular embodiments only, and is not intended to be limiting, since the scope of the present invention will be limited only by the claims. More particularly, the invention should not be limited to the specific details (unless required by the claim), such as particular machines, methods of infusion, components, anti-coagulants, techniques, protocols, systems, constituents, types of plasma, agents, reactants, proteins, donors, etc.

However, it will be apparent to one skilled in the art that the present invention may be practiced in other embodiments that depart from these specific details. Detailed descriptions of well-known anti-coagulants, solvents, proteins, thawers, apheresis machines, freezers, refrigerators, senolytics, medical procedures (e.g., donating plasma, plasma exchange, etc.) and the like are omitted so as not to obscure the description.

Where a range of values is provided, it should be understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art. Although any methods and materials similar or equivalent to those described herein can be used in the practice of the present invention, some potential and example methods and materials are described.

As will be apparent to those of skill in the art upon reading this disclosure, each of the individual embodiments described herein includes components and features which may be readily separated from or combined with the features or components of any of the other embodiments without departing from the scope or spirit of the present invention. Further, unless otherwise dictated by the context of the description, any recited method can be carried out in the order of events recited or in any other order which is logically possible.

Some embodiments of the present invention include kits, compositions and methods for treating a subject for one or more age-related conditions. Age-related condition is meant a condition, such as a disease or other undesirable condition, which accompanies aging of the species and/or in which aging is a significant or primary risk factor for the condition. The condition may manifest in the subject in a number of different ways including, but not limited to, aging associated damage to central or peripheral organs of the body, such as organ dysfunction, cell injury, tissue damage, shortening lifespan, cancer, where specific organs and tissues of interest include, but are not limited to skin, neuron, muscle, pancreas, brain, kidney, lung, stomach, intestine, spleen, heart, adipose tissue, testes, ovary, uterus, liver and bone. Some embodiments of the invention also include kits, compositions and methods for treating a subject to slow or reverse the effects of aging. As used herein, “to reverse the effects of aging” on an organism is meant to include transitioning the organism to a physiological state that the organism (or other organisms of the same species) experienced (or should have experienced or more than likely experienced) at an earlier time (i.e., at a chronological earlier date) in at least one organ (and/or tissue), such as two or more organs (or tissues), including three or more organs (and/or tissues), such as in four or more organs (and/or tissues), including at least five organs (and/or tissues) or in all organs (and/or tissues) for which measurements can be made. In other words, the organism, in effect, transitions to a biological younger physiological condition in some embodiments.

By “treatment”, “treating” and the like it is generally meant obtaining a desired pharmacologic and/or physiologic effect. The effect may be prophylactic in terms of completely or partially preventing a disease or symptom thereof and/or may be therapeutic in terms of a partial or complete cure for a disease and/or symptom or adverse effect attributable (directly or indirectly) to the disease (or aging). “Treatment” as used herein covers any treatment of a disease in a mammal, and includes: (a) preventing the disease from occurring in a subject which may be predisposed to the disease but has not yet been diagnosed as having it; (b) inhibiting the disease, i.e., arresting or slowing its development; or (c) relieving the disease, i.e., causing regression of the disease. Treatment may result in a variety of different physical manifestations, e.g., epigenetic changes, modulation in gene expression, rejuvenation of tissue or organs, etc. The treatment may be administered before or during the symptomatic stage of the disease, and in some cases after the symptomatic stage of the disease.

For example, some embodiments of the present invention may be used to treat subjects with a neurodegenerative condition such as Alzheimer's disease, Parkinson's disease, frontotemporal dementia, multiple sclerosis, Huntington disease, amyotrophic lateral sclerosis, myotonic dystrophy, vascular dementia, and the like. Some embodiments of the present invention alternately or additionally may be used to treat other age-related diseases such as atherosclerosis, cardiovascular disease, cancer, arthritis (rheumatoid or osteoarthritis), hypertension, orthostatic hypotension, frailty syndrome, urinary tract infection, hip fracture, bacteremia, urinary incontinence, osteoporosis, influenza, heart rhythm problems, hypothyroidism, pneumonia, eye disease (e.g., glaucoma, macular degeneration, cataracts), enlarged prostate, asthma, insomnia, fatigue, type 2 diabetes, and others.

Thus, treatment may result in cardiac output increases, blood pressure decreases, arteriosclerosis reduction, improved gas exchange by the lungs, an increase in vital capacity and faster expiratory flow rates, and increased creatinine clearance, reduced blood glucose and improved tone and elasticity of the skin atrophies, an increase in lean body mass, and lessening of degenerative changes in many joints. The treatment also may result in reversal of the effects of aging and one or more of these results, such as two or more, including three or more, four or more or at least five, may be evidence of reversal of the effects of aging in the subject. Other evidence of reversal of the effects of aging may be provided by Horvath's clock (DNA methylation) testing and/or telomere testing.

As discussed above, embodiments of the invention include treating a subject for age-related conditions. For example, some embodiments of the present invention may be used to treat age-related decline in peripheral organ function to provide rejuvenating and regenerative effects in muscle, liver, brain, heart, and pancreas. In some embodiments, the recipient's blood may benefit. Specifically, inflammatory factors, which typically increase with age may be reduced, while trophic factors, which typically decrease with age, may be increased. In some instances, embodiments of the present invention may result in epigenetic changes (changes in expression levels of one or more genes in one or more tissues of the host). The change in expression level a given gene may be greater than 0.25 fold, such as 0.5 fold or greater, such as 1.0 fold or greater, including 1.5 fold or greater. The tissue may vary, and in some instances may be muscle tissue, organ tissue (liver, brain, heart, lung, and pancreas, etc.) or nervous system tissue, e.g., central nervous system tissue, including brain tissue, e.g., hippocampal tissue. In some instances, the one or more genes whose expression is modulated, e.g., enhanced, is a gene encoding a product that is a member of a plasticity related signaling pathway (i.e., a synaptic plasticity regulation gene), e.g., TLR4, SDFR1, EGR1, Fos proteins, e.g., c-Fos, etc. In some instances, the modulation of hippocampal gene expression is manifested as enhanced hippocampal plasticity. In some instances, the one or more genes whose expression is modulated, e.g., enhanced, is a gene encoding a product that is a member of network related to synaptic plasticity and learning and memory, such as but not limited to: NTRK3, RELN, EPHA4, etc.

In some instances, treatment may result in an enhancement in the levels of one or more proteins in one or more tissues of the host. The change in protein level of a given protein may be 0.25 fold or greater, including 0.5 fold or greater, such as 1.0 fold or greater, including 1.5 fold or greater, where in some instances the level may approach that of a healthy wild-type level, e.g., within 50% or less, such as 25% or less, including 10% or less, e.g., 5% or less of the healthy wild-type level.

Young Donor Plasma

Embodiments of the present invention include methods, kits and compositions for reversing the effects of aging and for treating a subject suffering from an aging-related condition (e.g., an illness or condition such as those listed above wherein aging is a primary or significant risk factor) through the use of young donor blood plasma (hereinafter “young donor plasma” or “YDP”) product. In some embodiments, the methods include administering a YDP product to the subject and in some embodiments the methods further include removing a portion of the subject's blood plasma.

By a “plasma product” (and including YPD), it is meant any product derived from blood that comprises plasma other than whole blood. The term “plasma’ as is used refers to the pale-yellow liquid component of blood composed of about 92% water, 7% proteins such as albumin, gamma globulin, anti-hemophilic factor, and other clotting factors, and about 1% mineral salts, sugars, fats, hormones and vitamins. In some embodiments, the YDP product includes only plasma and does not contain any substantial red blood cells, white blood cells, or platelets wherein the content of each may be 5% or less, such as 1% or less, including 0.5% or less. Examples of blood products that comprise plasma that are suitable for use in the subject methods include blood products produced by filtering whole blood to remove white blood cells (“leukoreduction”) and/or other components, and blood product consisting essentially of purified plasma. In some embodiments, the YDP product that is employed is a non-whole blood plasma product in that the product is not whole blood and therefore lacks one or more components found in whole blood, such as erythrocytes, leukocytes, etc., at least to the extent that these components are present in whole blood. In some embodiments, the YDP product is substantially, if not completely, acellular, where the cellular content may be 5% or less, such as 1% or less, including 0.5% or less.

In some embodiments, plasma may be blood collected conventionally with EDTA at the anti-coagulant followed by centrifugation (or filtering) to separate the plasma. Prior to administration, the plasma may be dialyzed in PBS (phosphate-buffered saline) through a 3.5 kDa molecular weight exclusion membrane to remove EDTA and proteins having an average molecular weight of 3.5 kD or less.

Donors of YDP Product

As indicated above, the YDP product in embodiments herein may comprise a plasma product prepared from blood donated from young individuals. The blood may be drawn manually, with automated equipment, or with some combination thereof. Typically, the plasma is extracted via an apheresis machine as is well known in the art. Any volume may be drawn that does not endanger the life of the donor. In some instances, a volume of 200-600 milliliters of plasma is drawn, for example 300-550 ml, or 450-500 ml. The drawn blood may be treated with an agent that prevents coagulation, i.e., an anti-coagulant, (e.g., EDTA, citrate, oxalate, heparin, etc.). For example, the anti-coagulant may be added to the blood as it is drawn. As another example, the receptacle into which the blood is collected may comprise an anti-coagulant. Other agents, e.g., buffers, preservatives, e.g., phosphate, dextrose, adenine, glycerine, glucose, raffinose, etc., agents that kill viruses (e.g., solvent detergent), etc., may also be added to the blood product. In summary, in some embodiments only the plasma is donated. In other embodiments, whole blood is donated by the donor and the plasma is separated from the other constituents of the blood.

In various embodiments, the composition includes plasma from young donors under thirty-six years of age, more preferably under thirty-one years of age, still more preferably, under twenty-six years of age, still more preferably, under twenty-three years of age, and even more preferably, under twenty years of age, such as under eighteen years of age, including under 14 years of age, such as under ten years of age and under 5 years of age. The reference to “age” of the donor is meant to comprise the age of the donor at the time of donation. In yet another embodiment, the composition includes at least some plasma derived from umbilical cord blood. In one embodiment, the donors are between 16 and 22 years of age at donation and in another embodiment, donors are between 16 and 25 years of age at donation and in still another embodiment the donors are between 18 and 22 years of age at donation.

In some embodiments selected donors include men and women who have no more than one pregnancy. In another embodiment, donors include men and women who have never been pregnant. In yet another embodiment, the plasma is derived from only male donors. In other embodiments, donors (or only female donors) are tested (screened) for human leukocyte antigen (HLA) antibodies and/or neutrophil (human neutrophil antigen or HNA) antibodies to determine whether the antibodies of each (or either) are above a predetermined threshold and if they are, the donor's donated blood product (e.g., plasma) may be excluded from use (e.g., discarded).

In some embodiments, the YDP product (or a precursor thereof) may be tested for antibodies against human leukocyte antigen (HLA) (class I and class II) and human neutrophil antigen (HNA) and the antibodies may be removed or the plasma may be excluded from use. A variety of ELISA and flow cytometry based solid phase assays are available to test for HLA class I and class II antibodies. Screening for neutrophil antibodies requires the preparation of panels of fresh neutrophils and testing in agglutination, immunofluorescence, or flow cytometry assays.

In some embodiments, the YDP product, i.e., whole blood (prior to separation) or plasma-comprising fraction thereof, is processed to remove one or more polypeptide fractions, such as a polypeptide fraction having an average molecular weight below a predetermined threshold as described in U.S. Publ. No. 2015/0157664 (Appl. Ser. No. 14/562,401), which is hereby incorporated by reference in its entirety for all purposes. While the predetermined threshold may vary, thresholds of interest include, but are not limited to: 3.5 kDa, 10 kDa, 25 kDa, 50 kDa. In some instances, a specific proteinaceous component may also be removed such as, for example, cytokines, IgG or others. The phrase “average molecular weight” is meant to comprise the mass of a polypeptide as computed by multiplying the total number of amino acids in the polypeptide by the average molecular weight of 110 kD for each. Various methods are known in the art for the removal of polypeptides with a given molecular weight or less from liquid samples. For example, the blood product may be subject to size-exclusion chromatography (SEC), e.g., gel filtration chromatography, in which the plasma-comprising blood product is passed over a matrix of beads comprising pores that retard proteins of a given molecular weight or less, thereby depleting the flow-through of these small polypeptides. As another example, the blood product may be subjected to hydrodynamic chromatography (HDC), in which the parabolic or Poiseuille-like flow of a sample that develops under laminar flow through a tube or packed column causes larger particles to travel in the faster-moving flow at the center of the tube and smaller particles to be retarded along the slower-moving flow closer to the walls of the tube. Any convenient method, e.g., SEC, HDC, and the like, may be employed to remove proteins that have a given threshold average molecular weight or less from the blood product.

Specific fractions of interest that may be employed in various embodiments of the invention include, but are not limited to: fractions in which polypeptides having an average molecular weight of 3.5 kDa or less have been removed; fractions in which polypeptides having an average molecular weight of 10 kDa or less have been removed; fractions in which polypeptides having an average molecular weight of 25 kDa or less have been removed; fractions in which polypeptides having an average molecular weight of 50 kDa or less have been removed; and fractions in which polypeptides having an average molecular weight of any of the above thresholds (e.g., 3.5 kDa, 10 kDa, 25 kDa, 50 kDa) or less and IgG has been removed. In other words, the blood product may be viewed as a given molecular weight (e.g., 3.5 kD-; 10 kD-; 25 kD-; 50 kD-) depleted plasma-comprising blood product. In some embodiments, the fraction that is administered is not a denatured fraction.

The plasma of each donor may be screened in that a small sample of each donor's plasma is tested for various pathogens (e.g., HIV, Hepatitis B, C, nucleic acid test (NAT), etc.). The plasma that tests positive for any screened pathogens is discarded so that the young donor plasma (“YDP”) product is free from the pathogens.

The plasma (or blood of the donor) may also be tested for blood type to ensure a match with the recipient (when necessary).

The plasma (or the donor) may also be tested for various drugs such as cocaine, marijuana, heroin, Amphetamine, methamphetamine, angel dust/PCP, oxycodone, methadone, barbiturates, ecstasy, nicotine, benzodiazepine, etc. The plasma from donors that test positive for any screened drugs may be discarded so that the YDP product is free from the drugs. While plasma may be received from donors who are not paid, plasma may also be collected from donors who receive financial compensation (i.e., some type of financial benefit such as cash, gift card, etc.) for their plasma. Heightened screening (e.g., for drugs and/or pathogens) may be performed on donors who are paid for their plasma.

Agents

In some embodiments, the YDP product may be provided in conjunction with another agent having activity suitable to treat the age-related condition/illness and/or for treating aging Itself. For example, in some embodiments of the present invention, the YDP may be provided in conjunction with other agents such as one or more of: growth differentiation factor 11 (GDF11), Nicotinamide adenine dinucleotide (NAD) (and/or a precursor thereto), a senolytic (e.g., dasatinib, quercetin, fisetin or the combination of quercetin and dasatinib), metformin, rapamycin, low dosage lithium, one or more cytokines, testosterone, estrogen, human growth hormone (HGH), nitric oxide, Vitamin D3, Vitamin C, and/or Vitamin B12. A senolytic is meant to mean a class of molecules that can selectively induce death of senescent cells. Combinations of said agents may be administered orally before (e.g., weeks or days before), substantially immediately before, during or substantially immediately after the administering of the YDP product orally (e.g., hours or minutes before), sublingual, intravenously, or via another suitable method. Agents may be administer substantially immediately after, a day after, two to five days after, a week after, one to four weeks after or two months after the series of treatments (or each treatment). “Substantially immediately” is meant to mean within 24 hours or less, such as 12 hours or less, including 6 hours or less, 4 hours or less 2 hours or less, including one hour or less, such as thirty minutes or less. In other embodiments, the container holding the YDP product also includes the (added) agent therein and is administered along with the other components of the YDP via IV or during apheresis (or substantially immediately after completion of administering the YDP).

Thus, in some embodiments the kit 101 may comprise containers that, in addition to the plasma from the donor(s), includes an added quantity of an agent such as one or more proteins (e.g., GDF11) or other agent beyond what is present in the plasma of the donors. Cytokines are small proteins (typically about 5-20 kDa) that are important for cell signaling (e.g., carry signals from one cell to another) and may be used as an agent in some embodiments. Cytokines are crucial in immune responses such as fighting infections.

Thus, in some embodiments the agent may comprise a protein such as cytokines (including chemokines (e.g., Eoxtain, MIP)), growth factors (e.g., EGF, GM-CSF), enzymes, neurotrophins (nerve growth factor (NGF), NT-4, Brain-derived neurotrophic factor (BDNF), and the like as well recombinant proteins of the same. Ideally, the genes of the recombinant proteins come from humans, but may also come from other non-human species. The recombinant proteins may be produced via molecular cloning or polymerase chain reaction.

Embodiments of the present invention may also include reagents, devices and kits thereof for practicing one or more of the methods described herein. The reagents, devices and kits thereof may vary greatly. Reagents of interest include those mentioned herein with respect to the methods of preparing plasma-comprising blood product for transfusion into a subject in need thereof, for example, anti-coagulants, cryopreservatives, buffers, isotonic solutions, etc.

Kits

Kits comprising the YPP product, agents, reagents and/or cartridges (described below) are also envisioned. Kits may also comprise YDP product, blood/plasma collection bags, tubing, needles, centrifugation tubes, saline, and the like.

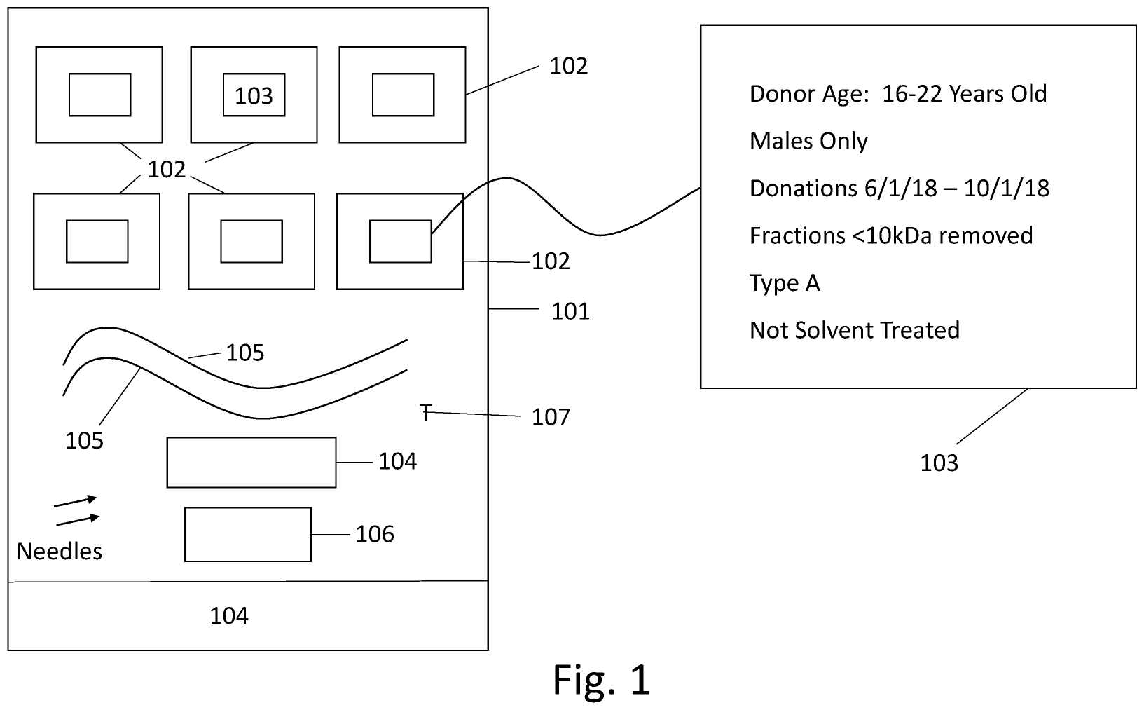

Referring to FIG. 1, the YDP product may be manufactured and included into kits. Each kit may be manufactured so that all of the plasma contained in a kit is from donors who at the time of donation are within a predetermined age range. For example, all the donors may have an age at donation of the plasma that is within 10 years or less of each other, including 7 years of each other or less, such as being within 5 years of each other or less, such as 4 years or less, or 3 years or less, such as two years or less including one year or less, including six months or less of each other. The kit may include indicia 103 (e.g., readable barcode, RFID integrated circuit, printed indicia, etc.) with information that relates to donor age such as the age window (i.e., the minimum and maximum age of the donors or permitted donors (e.g., (all donors 16-22 years old)), the average age of the donor of the donors at donation, the date(s) of donation, the mean age of the donors at donation, the age of the oldest donor at donation, the age of the oldest and youngest donors at donation. The information relating to donor age may alternatively include an age with a tolerance which accurately describes the age of all of the donors at the time of donation (e.g., 21 years+/−0.5, which would mean that all donors were within six months of 21 years old at their time of donation). The information relating to the YDP product also may include information related to the gender of the donor (male or female), race of the donor, the date(s) of a donation (such as date range of all donations of plasma in the kit), and processing details regarding the young plasma product, e.g., whether the plasma product was processed to remove proteins above an average molecule weight (such as described above), whether the YDP product was tested for antibodies, and/or whether the YDP product was processed to render viruses inactive (e.g., solvent-detergent treated plasma).

In some embodiments, kits may include two or more containers 102 of young donor plasma product, such as three or more, four or more, five or more, including six or more containers of young plasma product. In some instances, the number of distinct containers of young donor plasma product in the kit may be 9 or more, 12 or more, 15 or more, 18 or more, 21 or more, 24 or more 30 or more, including 36 or more, e.g., 48 or more.

The indicia 103 identifying information may be human readable and/or machine (e.g., computer, RFID, barcode) readable. The containers holding the YDP product may have varying configurations. While the volume of each container of embodiments may vary, in some embodiments the volumes range from 10 ml to 5000 ml, such as 25 ml to 2500 ml, e.g., 50 ml to 1000 ml, including 100 ml to 500 ml. The containers 102 may be rigid or flexible, and may be fabricated from any suitable material, such as steel, glass, polymeric materials, including medical grade plastic materials. In some embodiments, the containers 102 are of a bag configuration. In addition to the containers, such kits may further include administration devices, e.g., as described above. The components of such kits may be provided in any suitable packaging, e.g., a box or analogous structure, configured to hold the containers and other kit components. In one embodiment, the structure holding the containers may comprise insulation and in another embodiment may further include a cooling mechanism 104 sufficient to maintain FFP at a suitable (frozen) temperature for a predetermined duration in a range normal ambient temperatures.

The kit also may include indicia 103 with information identifying the blood type such as A, B, AB, or O (perhaps without regard to Rh factor). In some embodiments, all of the YDP product in a kit is of the same blood type. In other embodiments, the plasma may be of type AB and one other blood type (that of the recipient) so that it may be safely administered to a single recipient.

Blood Component ABO Compatibility Chart

| Patient's | Compatible Red | |
|---|---|---|
| ABO Group | Blood Cells (RBCs) | Compatible Plasma |
| Group O | Group O | Group O, A, B, AB |
| Group A | Group A and O | Group A and AB |
| Group B | Group B and O | Group B, AB |
| Group AB | Group O, A, B, AB | Group AB |

In many embodiments, the YDP product held in each container 102 is from a single donor, but the plasma in each container is from a donor different from the other donors. In yet another embodiment, each container in the kit includes plasma from the same donor. In other embodiments, the containers hold YDP product derived from pooled plasma. Pooled, as used herein, refers to plasma from multiple donors that is held in a container. In other words, the plasma from each donor is not maintained in a separate container but is co-mingled (and typically processed) with the plasma from other donors. In some embodiments the kit may comprise YDP product derived from pooled plasma.

Solvent detergent treated plasma is a form of blood plasma made from plasma which is then processed with solvents to render viruses inactive. Typically, solvent detergent treated plasma is pooled in that the plasma from multiple donors mixed (i.e., plasma from multiple donors share a container) and then processed although it is not necessarily pooled. Some embodiments of the present invention may comprise YDP product derived from solve detergent treated plasma.

If one or more agent(s) (such as those described above) are included in the same container as the YDP product, the kit may include information identifying the one or more agents and the amount such as the quantify, concentration or volume per container.

The amount of plasma in the kit 101 may be sufficient to treat the patient with the effective does one time or more times, such as two times, three times, 4 times or more, including 5 times or more.

The kits 101 may also include any other agents 104 (such as those described above) contained separately from the YDP product to be administered as well as the information of the identity, amount and, if applicable, the prescribed dosage of each agent. For example, one or more senolytics may be included.

In one embodiment, the YDP product comprises substantially only albumin, which is preferably at least 95% albumin or more, such as 97% albumin or more, including 98% albumin or greater, such as 99% albumin or more.

In other embodiments, the YDP product is preserved, e.g., by cryopreservation, etc., as known in the art until such time as when it is to be administered to a recipient. For example, a preparation may be frozen e.g., within about 24 or 48 hours of donation, i.e., immediately after collection (and processing if applicable) to about 48 hours after collection and stored at about −20° C. or less, e.g., −80° C. or less, in some instances −90° C. or less, or −135° C. or less, e.g., −196° C. As discussed herein, in some embodiments the blood preparation is fresh-frozen (is Fresh Frozen Plasma (FFP)). In other instances, a chemical preservative, e.g., a cryopreservative, e.g., dimethyl sulfoxide (DMSO), may be added to aid in preservation. Cryopreservatives find particular use in maintaining the viability of cells in the blood product, for example, if the plasma-comprising blood product also comprises leukocytes, erythrocytes, etc. For example, 20% or more of the cells will survive upon thaw, for example, 40% or more, 60% or more, 80% or more cells, in some instances, 90% or more, such as 95% or more, 97% or more, or 99% or more of the cells will be viable after removal of the preservative. The YDP product may be preserved prior to or after removal of proteins that are below a given threshold, such as described above, e.g., having an average molecular weight of 3.5 kD, 10 kD, 25 kD, 50 kD or less. In some embodiments, the YDP product may be preserved prior to the depletion or other processing. In other embodiments, the YDP product may be preserved after depletion or other processing. Following such techniques and/or techniques in the art, the YDP product may be stored for a year or more, e.g., 2, 3, 4, or 5 years or more, in some instances, 10, 20, 30 or 40 years or more, for example, 50, 60, 70 or 80 years. Upon thawing the YDP product, the preservative, if used, may be replaced with any convenient solution, e.g., any suitable isotonic solution, in preparation for administration to the subject.

Embodiments may include devices such as sterile cartridges, or columns, comprising a matrix that will retain or inhibit the flow of proteins having an average selected molecular weight such as a molecular weight of 3.5 kD or less. Such a cartridge may include (i) an inlet, (ii) a size exclusion matrix, (iii) an outlet; (iv) a housing that contains the matrix therein; and (v) a fluid path through the housing that connects the inlet to the outlet. The cartridge may comprise a support comprising at least one fluid-permeable membrane, one or more porous fiber(s), or a plurality of particles. The support may be formed separately from the housing or as an integral part thereof, and may be manufactured from any convenient material include, for example, alumina, cellulose, dextran, polyacrylamide, polyacrylate, polyamide, or silica. The cartridge may be configured for separation by membrane filtration or column chromatography, and may be able to bind from about a suitable amount of protein, e.g., grams of protein. Aseptic packaging may surround the cartridge to maintain it, the inlet, and the outlet in sterile and pyrogen-free conditions. The housing may be sized to comprise a volume of about 200-500 ml although larger cartridges also may be used. Cartridge parts (e.g., inlet, outlet, and housing) may be manufactured from glass, polypropylene, polystyrene, or stainless steel. An external pump may provide line pressure through flexible tubing to the cartridge and, thereby, control the flow rate of a fluid phase through the matrix and column.

In some embodiments, the kit 101 may further include one or more needles (e.g., one for removal and one for replacement of plasma), tubes 105 for connecting the needles to the apheresis machine, saline (bagged for administering to the subject), an intravenous T connector (e.g. for administering an agent), bandages, calcium carbonate (to mitigate the effects of the anticoagulant (hypocalcemia)), and empty container(s) 106 (e.g., bags) for receiving plasma removed from the subject. FIG. 1 illustrates an example kit 101 with six bags 102 of plasma, two tubes 105, two needles, six empty bags 106, a senolytic agent 104, a T connector 107 held in container that includes a cooling mechanism 104.

Selecting YDP Product

As discussed, embodiments of the present invention may be for treating aging individuals suffering from or at risk of suffering from one or more age-related conditions or illnesses. An aged human subject, as used herein, is meant include an individual that is about 40 years or older such as subjects 50 years old or older, including 60 years old or older, e.g., 70 years old or older, 80 years old or older, such as 90 years old, i.e., between the ages of about 50 and 100, e.g., 50, 55, 60, 65, 70, 75, 80, 85 or about 90 years old, and suffers from one or more age-related conditions or illnesses including those described herein or others.

In some embodiments, the YDP product may be selected based on the parameters of the YDP product such as the age of the donors at donation, or the age of the donors at donation relative to the age of the subject recipient. For example, the age of the recipient relative to the age of the donors at donation in one embodiment may be at least three so that a sixty year old recipient would receive YDP product from donors who were age twenty (or younger) at donation (because the ratio of 60 to 20 is 3). In other embodiments, the ratio (between the age of the recipient and the donor at donation) may be at least two, such as 2.5 or more, such as three or more, including 3.5 or more such as 4 or more, including 5 or more. If the donors are not all the same age at the time of donation, in one embodiment the ratio uses the average age of the donors at donation, in other embodiments the ratio uses the mean age of the donors at donation, in still other embodiments the ratio uses the maximum permitted age of the donors at donation.

While the donor of the YDP product may be different from that of the aging subject recipient, there may be benefits to using donors that are genetically similar to the reception. Consequently, in other embodiments, an individual's plasma or YDP product derived from that individual's plasma may be preserved (e.g., such through cryopreservation as fresh frozen plasma) and subsequently used to treat the individual when a need arises (perhaps decades later). In other embodiments, plasma from a recipient's children, siblings, grandchildren, grandparents, cousins, or other descendants may be used to produce the YDP product to treat the recipient.

In some embodiments, characteristics of the donors may be used to select the YDP product for a given recipient. For example, in one embodiment parameters of the YDP product such as the donors' race may be selected to match the race of the recipient. Alternately, or additionally, parameters of the YDP product such as the donors' gender may be selected to match the gender of the recipient

In some embodiments, the YDP product to be administered is selected based upon the blood type of the donor(s) and the blood type of the recipient. By blood type, it is meant the presence or absence of A and B antigens (and in some embodiments the Rh factor) on the donor and recipient's red blood cells. In other embodiments, such as when the blood product is a fractionated product that comprises no cells displaying the NB or Rh antigens, for example, a blood product that consists essentially of plasma, the blood product from a donor of any blood type may be administered to the subject.

Administering

The YDP product may be administered using any convenient protocol for administering blood product to an individual. In some instances, the YDP product may be administered intravenously. The YDP product may be mixed with intravenous solutions as is known in the art, e.g., 5% dextrose in water, an isotonic electrolyte solution such as isotonic saline (0.9%), etc. The YDP product may be administered using any convenient access device, e.g., needle for intravenous injection, compressor gun, peripheral cannula, central IV line, etc., e.g., implantable port, tunneled line, central venous lines, peripherally inserted central catheters and the like. In various embodiments, administration may be through any vein typically used for transfusion, e.g., subclavian, internal jugular, femoral, superior vena cava, inferior vena cava, right atrium, etc., in a volume and at a rate typically used for transfusion as known in the art, e.g., 10-20 ml per Kg weight of the individual per dose, at a rate of about 5 ml per minute.

In some embodiments, the YDP product may be infused into the patient one or more times (over one or more days) such as via an intravenous (IV) drip. In other embodiments, the YDP product may be administer as part of one or more plasma exchanges (over one or more days) in which a portion of the subject's plasma is removed and replaced with the YDP product.

In a plasma exchange embodiment, a practitioner will insert a needle attached to a catheter (i.e., a tube) into a vein in each arm. If the subject's arm veins are too small, the needle may need to be placed the shoulder or groin instead. The subject's blood leaves the subject via one of the tubes and travels to an apheresis machine that separates the plasma from other blood cells as is well known in the art. The other blood cells are then mixed with the YDP product and the new blood YPD product mixture travels into the patient through the second tube.

In some embodiments, the apheresis machine separates the plasma from the other blood cells via centrifugation while in other embodiments filtering is used.

The prepared YDP product may be administered to the subject administered immediately, e.g., within about 12-48 hours of collection. In such instances, the blood product may be stored under refrigeration, e.g., 0-10° C. and warmed prior to administering.

The effective amount of YDP product to be administered may vary depending on the composition of the YDP product to be administered, the method of administration, the age of the recipient (in some instances), the size and gender of the recipient, and the nature of the condition that is being treated. In some embodiments, the YDP product may be administered once. In other instances, the YDP product may be administered more than once, e.g., regularly, such as weekly, monthly, biannually, or annually. For example, the YDP product may be administered weekly for 2 weeks or more, e.g., 3 weeks, 4 weeks, 5 weeks, 6 weeks, 7 weeks, 8 weeks, more than 8 weeks, etc. As another example, the YDP product may be administered monthly, e.g., for 2 months or more, e.g., 3 months, 4 months, 5 months, 6 months, 7 months, 8 months, 9 months, 10 months, 11 months, 12 months, or more than 12 months. It will be understood by those of skill in the art that an initial dose may be administered for such periods of time, followed by maintenance doses, which, in some cases, may be at a reduced dosage.

In one embodiment, the treatment is administered five times with treatments occurring every three to five days (e.g., being separated by at least two days but no more than four days such as on each Monday and Thursday for five treatements). In other embodiments, the treatment is performed only once, three times, seven times, and ten times. Further, in other embodiments, the series of treatments occur every other day, every three to seven days, every five to seven days, every seven to ten days, and every ten to fourteen days. In still other embodiments, the treatment is performed once monthly, once quarterly, once every six months, and annually.

In various embodiments, the subject receiving the treatment is administered an effective amount of the YDP at one or more treatment times. An effective amount, or dose, of YDP product is an amount that, when administered over a suitable period of time, will evidence a reduction in the rate of decline of the condition of the subject, a stabilization of the condition of subject (e.g., no further decline), or a reversal (improvement) in the condition (e.g., a cure) of the subject. For example, an effective dose is the dose that, when administered for a suitable period of time, such as at least about one week, and maybe about two weeks, or more, up to a period of about 3 weeks, 4 weeks, 8 weeks, or longer, will slow e.g., by about 20% or more, e.g., by 30% or more, by 40% or more, or by 50% or more, in some instances by 60% or more, by 70% or more, by 80% or more, or by 90% or more, e.g., will halt the age-related condition or illness of the subject.

An “effective amount” or “effective dose” of blood product to prevent or treat an aging related condition it is meant an amount of YDP product that is administered one or more times that will inhibit, antagonize, decrease, reduce, or suppress by about 20% or more, e.g., by 30% or more, by 40% or more, or by 50% or more, in some instances by 60% or more, by 70% or more, by 80% or more, or by 90% or more, in some cases by about 100%, i.e., to negligible amounts, and in some instances reverse, the age-related condition. An “effective amount” or “effective dose” of YDP product to reverse one or more effects of aging is meant to mean an amount of YDP product that when administered one or more times will transition the organism to a biological younger physiological state. In other words, it is a dose sufficient to transition the organism to a physiological state that the organism experienced at an earlier time (i.e., at a chronological earlier date) in at least one organ (and/or tissue), such as two or more organs (or tissues), including three or more organs (and/or tissues), such as in four or more organs (and/or tissues), including at least five organs (and/or tissues) or in all organs (and/or tissues) for which measurements can be made.

The amount of YDP product to be administered to the subject may be based on the subject's plasma volume. The subject's plasma volume may be computed, measured, or estimated using any conventional means such as:

EPV=(0.065×wt (kg))×(1−Hct).

Wt=the subject's weight

Hct=hematocrit

Another estimate of plasma volume may be computed based on body weight as 30 mL/kg of plasma.

In one embodiment, one plasma volume of YDP product is administered. In other embodiments, approximately a half plasma volume, three quarter plasma volumes, one and a quarter plasma volumes, and one and a half plasma volumes (or more), such as two plasma volumes are administered at each treatment.

In one embodiment, a greater volume of plasma is removed from the patient than the amount of YDP product that is administered to the subject. For example, one and half plasma volumes of YDP product may be administered and only one volume (or 1.25 volumes) of plasma removed.

In one embodiment, a lesser volume of plasma is removed from the patient than the amount of YDP product that is administered to the subject. For example, one half plasma volume of YDP product may be administered and one plasma volume of plasma removed.

In one embodiment, the treatment is administered to the patient within two hours of the subject waking. In other embodiments, the treatment is administered to the subject within four hours, six hours, and eight hours of the subject waking.

In one embodiment, the anti-coagulant used during treatment (for plasma exchange embodiment) is sodium citrate. In other embodiments, the anti-coagulant is EDTA (Ethylenediaminetetraacetic acid), citrate, oxalate, or heparin.

A one plasma volume exchange removes about 66% of an intravascular constituent and a two volume plasma exchange removes approximately 85%. Consequently, a significant portion (approximately one third) of the replacement fluid supplied during the beginning of the exchange is removed. Thus, in some embodiments, saline (or another substance) may used as the replacement fluid for at least the first ten percent of the exchange, including the first fifteen percent or more, such as the first 20% or more, including the first 25% or more. As an example, where one volume of plasma is to be removed, while the first 20% of the subject's plasma is removed, the replacement fluid administered to the subject may be saline. The replacement fluid may then be switched to the YDP product so that the next 80% of plasma that is removed (the remainder) is replaced with the YDP product.

In accordance with one example embodiment, the YDP product is derived from young donors who are aged 16-22 at donation of their plasma. Their plasma is donated via a plasma donation (as opposed to whole blood donation) as is well known in the art and EDTA used as the anti-coagulant. In this embodiment, all donors are males. The plasma is freshly frozen (within 24 hours) to provide FFP. The plasma of each donor is screened in that a small sample of each donor's plasma is tested for various pathogens (e.g., HIV, Hepatitis B, C, etc.). The plasma that tests positive for any screened pathogens is discarded so that the YDP product is free from the pathogens.

In this embodiment, the recipient subject is at least 50 years of age and may be suffering from one or more age-related conditions or illness. The subject receives a series of five plasma exchanges in which his or her plasma is removed and replaced with the YDP product. In this example embodiment, one volume of plasma is removed and one volume of YDP product is administered at each exchange. The plasma exchanges occur every 3-5 days such as at each Tuesday and Friday. Thus, the kit 101 in this example may include five volumes of YDP product (as estimated for the specific patient) in order to complete the five plasma exchanges. Alternately, the kit may include one volume of YDP product and one kit used for each exchange. The series of plasma exchanges (five in this embodiment) may be repeated every six months, annually or every two years.

In this embodiment, plasma removed from the patient may be stored and processed for subsequent use to provide FFP or a plasma derivative (although in this embodiment it would not be considered a YDP product).

In accordance with a second example embodiment, the YDP product is derived from young donors who are aged 16-25 at donation of their plasma. Their plasma is donated via a plasma donation (as opposed to whole blood donation) as is well known in the art and EDTA used as the anti-coagulant. In this embodiment, all donors are females who report never having been pregnant. The plasma is freshly frozen (within 24 hours) to provide FFP. The plasma of each donor is screened in that a small sample of each donor's plasma is tested for various pathogens (e.g., HIV, Hepatitis B, C, etc.). The plasma that tests positive for any screened pathogens is discarded so that the YDP product is free from the pathogens.

In this embodiment, the recipient subject is at least 50 years of age, a female, and may be suffering from an age-related condition or illness. The subject receives a series of seven plasma exchanges in which her plasma is removed and replaced with the YDP product. In this example embodiment, 1.5 volumes of plasma are removed and 1.5 volumes of YDP product administered at each exchange. The plasma exchanges occur once per week. Thus, the kit in this example may include 7.5 volumes of YDP product (as estimated for the specific patient), derived only from females, in order to complete the five plasma exchanges. Alternately, the kit may include only enough plasma and other items for completing one exchange. The series of plasma exchanges (7 in this embodiment) may be repeated every six months, annually or every two years. In this embodiment, plasma removed from the patient may be stored and processed for subsequent use to provide FFP or a plasma derivative (although in this embodiment it would not be considered a YDP product).

In one embodiment, a kit is provided comprising two or more containers, wherein each container comprises a young donor plasma product and identifying information comprising age related data of the young donor plasma product. The young donor plasma product is sourced from a plurality of donors and wherein said plurality of donors are within an age range at the time of donation and wherein the age range includes an upper limit of 25 years of age or less; and wherein the age range includes a lower limit of 16 years of age or more.

In one embodiment, the age range has an upper limit of twenty-two years of age and/or the young donor plasma product is sourced from only male donors.

In one embodiment, the young donor plasma product is sourced from donors all having the same blood type without regard to Rh factor.

In one embodiment the young donor plasma product is sourced from donors all having type AB blood and only one other blood type.

In one embodiment, the young donor plasma product is pooled such as from all male donors of a given age range. In another embodiment each container comprises young donor plasma product sourced from a single donor and each container is sourced from a different donor.

In one embodiment, the kit further comprise a container of GDF-11.

In another embodiment, the each container includes an added quantity of GDF-11 beyond what is present in the plasma of the donors.

In one embodiment, the young donor plasma product comprises substantially albumin.

In one embodiment, the young donor plasma product includes donors that at donation were no more than one half (or alternately one third) the age of an intended recipient.

In one embodiment, the kit further comprises a container having a quantity of a senolytic.

In one embodiment, the young donor plasma product has been processed to remove fractions having an average molecular weight below a predetermined threshold.

Another embodiment includes a kit, comprising two or more containers, wherein each container comprises a young donor plasma product; and Indicia with information comprising age data of the young donor plasma product, blood type information, and collection date information. The young donor plasma product is sourced from a plurality of donors and the plurality of donors are within an age range at the time of donation. The age range includes an upper limit of 25 years of age or less and a lower limit of 18 years of age or more; and wherein the young donor plasma product is sourced entirely from male donors.

In another embodiment, the age range has an upper limit of twenty-two years of age. In another embodiment the young donor plasma product is sourced from donors all having type AB blood and only one other blood type without regard to Rh factor.

In another embodiment the kit further comprises a container of GDF-11.

In yet another embodiment, the young donor plasma product includes donors that at donation were no more than one half the age of an intended recipient.

In one embodiment, the young donor plasma product has been processed to remove fractions having an average molecular weight below a predetermined threshold.

In still another embodiment, the kit, comprises two or more containers, wherein each container comprises a young donor plasma product information comprising age related data of the young donor plasma product and wherein the young donor plasma product is sourced from a plurality of donors and wherein said plurality of donors are within an age range at the time of donation and wherein the plurality of donors of the young donor plasma are all male and wherein the age range includes an upper limit of 25 years of age or less wherein the age range includes a lower limit of 16 years of age or more.

In this embodiment, the young donor plasma product has been processed to remove fractions having an average molecular weight below a predetermined threshold.

Another embodiment of the present invention, comprises a method of reversing the effects an aging in a subject, the method comprising administering to a subject an effective amount of a young donor plasma product to reverse one or more effects of aging in the subject, wherein said administering transitions the subject to a biological younger physiological condition. The method may comprise administering to the subject an effective dose of young donor plasma on a first treatment day, administering to the subject an effective does of young donor plasma on a second treatment day; wherein said second treatment day is at least three days after said first treatment day and no more than seven days after said first treatment day; administering to the subject an effective does of young donor plasma on a third treatment day; and wherein said third treatment day is at least three days after said second treatment day and no more than seven days after said second treatment day. The doses, for example, may comprise one volume of plasma and be administered via plasma exchange or IV drip (dosing a smaller amount).

It is to be understood that the foregoing illustrative embodiments have been provided merely for the purpose of explanation and are in no way to be construed as limiting of the invention. Words used herein are words of description and illustration, rather than words of limitation. In addition, the advantages and objectives described herein may not be realized by each and every embodiment practicing the present invention. Further, although the invention has been described herein with reference to particular structure, materials and/or embodiments, the invention is not intended to be limited to the particulars disclosed herein. Rather, the invention extends to all functionally equivalent structures, methods and uses, such as are within the scope of the appended claims. Those skilled in the art, having the benefit of the teachings of this specification, may affect numerous modifications thereto and changes may be made without departing from the scope and spirit of the invention.

What is claimed is:

1. A kit, comprising:
two or more containers, wherein each container comprises:
a young donor plasma product, and
indicia with information related to age of one or more donors of the young donor plasma product;
wherein the young donor plasma product is sourced from a plurality of donors;
wherein said plurality of donors are within an age range at the time of donation;
wherein the age range includes an upper limit of 25 years of age or less; and
wherein the age range includes a lower limit of 16 years of age or more.

2. The kit of claim 1 wherein the young donor plasma product includes donors that at donation were no more than one third the age of an intended recipient.

3. The kit of claim 1, wherein the age range has an upper limit of twenty-two years of age.

4. The kit of claim 1 wherein the young donor plasma product is sourced from only male donors.

5. The kit of claim 1 wherein the young donor plasma product is sourced from donors all having the same blood type without regard to Rh factor.

6. The kit of claim 1 wherein the young donor plasma product is sourced from donors all having type AB blood and only one other blood type.

7. The kit of claim 1 wherein each container comprises young donor plasma product sourced from a single donor.

8. The kit of claim 1, further comprising a container of GDF-11.

9. The kit of claim 1, wherein each container includes an added quantity of GDF-11 beyond what is present in the plasma of the donors.

10. The kit of claim 1 wherein the young donor plasma product comprises substantially albumin.

11. The kit of claim 1 wherein the young donor plasma product includes donors that at donation were no more than one half the age of an intended recipient.

12. The kit of claim 1, further comprising a container having a quantity of a senolytic.

13. The kit of claim 11, wherein the young donor plasma product has been processed to remove fractions having an average molecular weight below a predetermined threshold.

14. A kit, comprising:
two or more containers, wherein each container comprises:
a young donor plasma product; and
indicia with information comprising:
age related data of the donors of young donor plasma product,
blood type information, and
collection date information;
wherein the young donor plasma product is sourced from a plurality of donors;
wherein said plurality of donors are within an age range at the time of donation;
wherein the age range includes an upper limit of 25 years of age or less;
wherein the age range includes a lower limit of 18 years of age or more;
wherein the young donor plasma product is sourced entirely from male donors.

15. The kit of claim 14, wherein the age range has an upper limit of twenty-two years of age.

16. The kit of claim 14, wherein the young donor plasma product is sourced from donors all having type AB blood and only one other blood type.

17. The kit of claim 14, further comprising a container of GDF-11.

18. The kit of claim 14, wherein the young donor plasma product includes donors that at donation were no more than one half the age of an intended recipient.

19. The kit of claim 14, wherein the young donor plasma product has been processed to remove fractions having an average molecular weight below a predetermined threshold.

20. A kit, comprising:
two or more containers, wherein each container comprises:
a young donor plasma product;
indicia comprising information of related to age of donors of the young donor plasma product;
wherein the young donor plasma product is sourced from a plurality of donors;
wherein said plurality of donors are within an age range at the time of donation;
wherein the plurality of donors of the young donor plasma are all male;
wherein the age range includes an upper limit of 25 years of age or less;
wherein the age range includes a lower limit of 16 years of age or more; and
wherein the young donor plasma product has been processed to remove fractions having an average molecular weight below a predetermined threshold.
